(19) United States
(12) Patent Application Publication
(10) Pub. No.: US 2013/0199271 A1
(43) Pub. Date: Aug. 8, 2013

(54) METHOD AND DEVICE FOR DETECTING EXPLOSIVE-SUBSTANCE PARTICLES IN A GAS FLOW

(71) Applicants: Sebastian Beer, Regensburg (DE); Thomas Ziemann, Inning am Holz (DE); Alois Friedberger, Oberpframmern (DE)

(72) Inventors: Sebastian Beer, Regensburg (DE); Thomas Ziemann, Inning am Holz (DE); Alois Friedberger, Oberpframmern (DE)

(73) Assignee: EADS DEUTSCHLAND GMBH, Ottobrunn (DE)

(21) Appl. No.: 13/809,413

(22) PCT Filed: Jun. 17, 2011
(86) PCT No.: PCT/DE2011/001309

§ 371 Date: Apr. 16, 2013

(30) Foreign Application Priority Data
Jul. 13, 2010 (DE) 10 2010 027 074.1

(51) Int. Cl. G01N 1/40; G01N 33/00

(52) U.S. Cl. CPC G01N 1/4005; G01N 33/0057
USPC 73/28.04; 430/320

(57) ABSTRACT

A method for detecting explosive substance particles in a gas flow includes passing the gas flow through an adsorption net for a specified time period so as to adsorb explosive-substance particles in the gas flow on the adsorption net. The adsorption not includes a microfilter having a pore size that is smaller than the particle size of the explosive-substance particles. The adsorption net is heated to a heating temperature so as to desorb the explosive-substance particles from the adsorption net. A gas flow comprising the desorbed explosive-substance particles is supplied to a detector so as to detect the explosive-substance particles.

### CROSS REFERENCE TO RELATED APPLICATIONS

This application is a U.S. National Phase application under 35 U.S.C. §371 of International Application No. PCT/DE2011/001309, filed on Jun. 17, 2011, and claims benefit to German Patent Application No. DE 10 2010 027 074.1, filed on Jul. 13, 2010. The International Application was published in German on Jan. 26, 2012, as WO/2012/010123 A1 under PCT Article 21 (2).

### FIELD

The invention relates to a method and a device for detecting explosive-substance particles in a gas flow, in which the gas flow is conducted through an adsorption net for a specified time period, in such a way that explosive-substance particles are adsorbed thereon, the adsorption net is subsequently heated to a heating temperature, at which the explosive-substance particles desorb, and a gas flow containing the accumulated explosive-substance particles is supplied to a detector for detection thereof.

### BACKGROUND

A detection method and a detection device are known from U.S. Pat. No. 6 604 406 B1.

The increasing use of explosive substances for the purposes of terrorism, in particular in civilian air transport, creates an urgent need for efficient explosive-substance detectors, systems which are portable or suitable for use in the field being necessary in particular. If for example a potential terrorist processes an explosive substance, this leaves behind small explosive-substance traces on clothing and skin. The purpose of a detection method for explosive-substance traces is to discover these explosive-substance traces, for example before entry to an aeroplane. In this context, a gas flow, generally ambient air, is passed over an article or a person to be analysed, explosive-substance particles being carried along if present. However, this type of detection is made difficult by the very low concentrations of the explosive substances, which are often in the ppt range (parts per trillion), direct detection of the explosives in the gaseous phase being very difficult in some cases since the equilibrium gas concentrations of conventional explosive substances are very low.

A detection method for explosive substances is disclosed in U.S. Pat. No. 6,604,406, in which the substances to be searched for are collected as particles on an adsorption net in the form of a felt, non-woven or mesh and subsequently supplied to a detector. In this previously known method, in a first adsorption step, the gas which contains explosive-substance particles at a low concentration is sucked through the adsorption net in the form of felt, non-woven or mesh, some of the particles being adsorbed on the filter and the concentration of particles on the filter thus increasing over time. In a second method step, the desorption step, the adsorption net is heated and the flow direction of the gas flow through the adsorption net is reversed. In this context, the accumulated explosive-substance particles are desorbed from the adsorption net and can be detected by the detector at an increased concentration. A drawback in this context is that only relatively large particles remain suspended in the absorption net, whilst the smaller particles pass through and thus cannot contribute to the detection.

### SUMMARY

In an embodiment, the present invention provides a method for detecting explosive substance particles in a gas flow including passing the gas flow through an adsorption net for a specified time period so as to adsorb explosive-substance particles in the gas flow on the adsorption net. The adsorption net includes a microfilter having a pore size that is smaller than the particle size of the explosive-substance particles. The adsorption net is heated to a heating temperature so as to desorb the explosive-substance particles from the adsorption net. A gas flow comprising the desorbed explosive-substance particles is supplied to a detector so as to detect the explosive-substance particles.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 shows a first embodiment of the device for detecting explosive-substance particles;

FIGS. 2a and 2b shows a second embodiment of the device for detecting explosive-substance particles in two different operating states;

FIG. 3 shows a third embodiment of the device for detecting explosive-substance particles;

FIGS. 4a and 4b shows two embodiments of detection devices having heatable microfilters.

### DETAILED DESCRIPTION

An aspect of the present invention is to improve the detectability of explosive substances further or to reduce the detection threshold further.

In an embodiment, the present invention provides the use of a microfilter, of a pore size which is smaller than the particle size or the particle diameter of the explosive-substance particles to be detected, as an adsorption net. In this context, the term “microfilter” is understood to mean a membrane of a thickness in the range of approximately 1 μm, which has mechanical stability as a result of support structures and comprises regular perforations. These perforations are preferably of an identical diameter, which is preferably smaller than 1 μm, more preferably smaller than 400 nm. This makes it possible, unlike in the prior art, for all of the particles located in the gas stream to be captured or adsorbed thereon, whereas in conventional systems a significant proportion of the particles can pass through the mesh of the adsorption net, in such a way that the accumulation is much weaker or takes much longer. By means of the microfilter, the particles can be retained on the surface, and as a result they remain easily accessible and can easily be desorbed again. By contrast, the conventional meshes are three-dimensional fabrics or felts. This construction according to the invention is advantageous in particular in the desorption step, since all of the particles are located on a single surface and not in a three-dimensional structure, and targeted desorption is thus possible by heating the microfilter surface. This targeted heating to predetermined temperatures can further be used so as to achieve detection selectivity for particular explosive substances by setting particular temperatures.

In this way, portable particle—gas conversion of small explosive-substance particles can advantageously be made possible. The low thermal mass of the microfilter makes low-power operation possible along with a very rapid temperature increase during the heating process. In this way, instead of the particles merely being desorbed, they could also be dissociated, molecule groups being split off, and this would make alternative detection options possible, for example tracing molecules comprising nitrogen groups.

The pore size of the microfilter is preferably selected as a function of the explosive substances to be detected, in such a way that it is also possible to use microfilters of different pore sizes to detect particular explosive substances. It is also possible to make the microfilter replaceable for this purpose.

So as further to increase the selectivity of the detection, it is also possible to arrange two microfilters of different pore diameters in succession, the first microfilter having a larger pore size (for example 1 μm) so as to capture large, undesired particles, and a second microfilter of a smaller pore size (for example 400 nm) being provided downstream, on which the particles to be detected are adsorbed. In the second method step, only the second microfilter is heated, in such a way that only the explosive-substance particles adsorbed thereon are desorbed and supplied to the detector. Subsequently, after the end of the detection process, the first filter can also be heated so as to remove the undesired particles adsorbed thereon.

In accordance with an advantageous development of the invention, a heating temperature is set and a microfilter is used of a pore size at which the explosive-substance particles can pass through the microfilter in the gaseous phase after the heating and desorption. This temperature is approximately 150° to 250°. In this particularly simple embodiment of the method, which can also make use of a device of a simple construction, it is not necessary for gas to flow through the arrangement in different flow directions. In this context, the gas flow is preferably permanently activated, the microfilter being flowed through permanently and the gas detector being flowed over constantly by the gas flow. However, after a particular time (in particular approximately 10-20 s), when enough particles have been absorbed on the microfilter and the microfilter is heated, there is a sufficient concentration of explosive-substance particles, which can be detected well by the detector, in the resulting desorption of the accumulated explosive-substance particles.

A preferred device for carrying out the aforementioned method comprises a microfilter, downstream from which a detector is arranged, the microfilter comprising a heating device and a control device for controlling the temperature of the microfilter. In this simple arrangement, the microfilter and the detector are always flowed through in the same direction by the gas flow comprising the explosive-substance particles, and this is very simple in terms of construction.

An alternative development of the method according to the invention provides that, in a collection mode, the gas flow is passed through the microfilter, and then in a subsequent detection mode, a gas flow flows through the microfilter, which is warmed in the process, in the reverse flow direction. In this context, the explosive-substance particles adhering to the microfilter are desorbed, and can be analysed in this accumulated form in the detector. In this context, the gas flow is circulated in a closed circuit in the detection mode.

A device for carrying out this embodiment of the method comprises a flow duct having a microfilter and a circulation duct having a detector, which can be blocked off in the collection mode and can be connected to the flow duct in the detection mode so as to form a closed annular duct.

In accordance with an advantageous development, the device comprises a halogen lamp for heating the microfilter, it being possible either to achieve parallel, uniform irradiation of the whole microfilter by using a collimator or to achieve a targeted orientation onto particular regions of the filter by means of focussing lenses. In conjunction with an optical or resistive thermometer, the temperature of the microfilter can be measured precisely making it possible to set a particular temperature in a targeted manner. This makes it possible to set particular predetermined temperature progressions over time, allowing selectivity to be achieved for different types of explosive substance.

A method for producing a microfilter for using one of the prescribed devices is preferably produced by a photolithography etching process, making it possible to form all of the pores of the microfilter at an identical diameter in the desired size range.

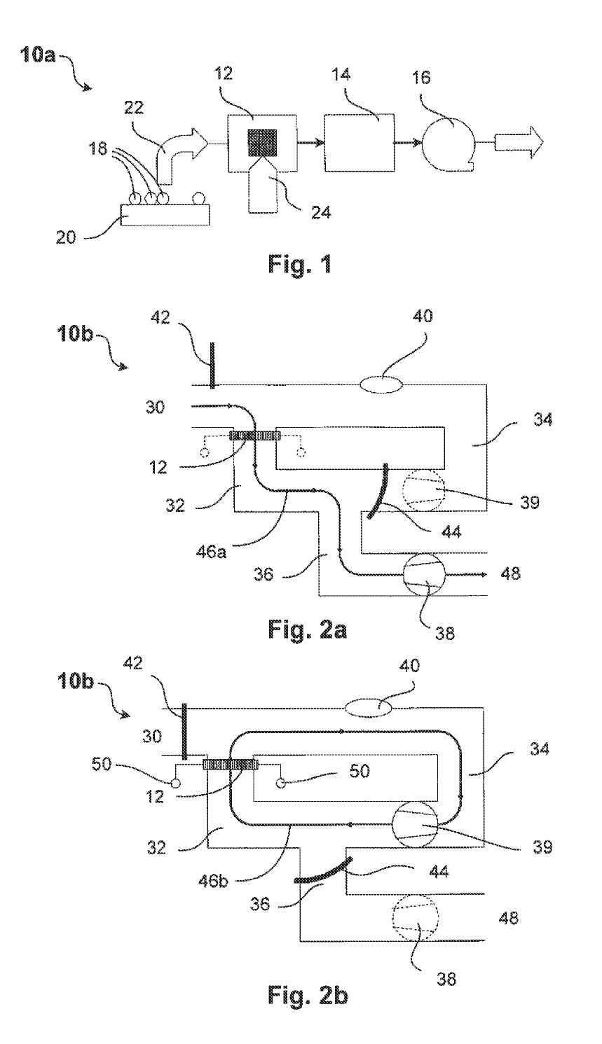

FIG. 1 shows schematically a first embodiment of a detection device 10a, which basically consists of a microfilter 12, a detector 14 and a suction pump 16. An article 20 contaminated with explosive-substance particles 18 is also shown schematically, over which an air flow 22 is passed, which flows through the microfilter 12 and further passes through the detector 14. In this context, the explosive-substance particles 18 (shown greatly enlarged in the drawings) adhere to the microfilter 12, since they cannot pass through the microfilter 12 as a result of the selected pore size thereof, which is smaller than the size of the explosive-substance particles 18. After a particular time, preferably approximately 10 to 20 s, enough explosive-substance particles 18 have accumulated on the microfilter 12, and so the microfilter 12 is heated by means of the heating device 24, preferably to a temperature of approximately 150 to 250° C. As a result of the increased temperature, the explosive-substance particles 18 are desorbed from the microfilter 12 and enter into the gaseous phase, in which they can pass through the pores of the microfilter 12 and can thus be supplied to the detector 14 at an increased concentration. After a particular period of a few seconds, within which substantially all of the explosive-substance particles 18 adhering to the microfilter 12 are desorbed, the heating device 24 is switched off again, and a further article 20 to be analysed can be analysed for explosive-substance particles 18, again by means of a gas flow 22.

FIGS. 2a and 2b show schematically a second embodiment of a device 10b for detecting explosive-substance particles. This comprises a gas inlet 30, to which a flow duct 32 is attached, in which a microfilter 12 is arranged. The flow duct 32 is connected at one end to a U-shaped circulation duct 34, which is connected to the flow duct 32 on both sides of the microfilter 12. The flow duct 32 is further connected to an outlet duct 36, in which a suction pump 38 is arranged. A circulation pump 39 is arranged in the circulation duct 34. A detector 40 is further arranged in the wall of the circulation duct 34, and is preferably an ion mobility spectrometer (IMS) or a metal oxide semiconductor gas sensor (MOX sensor). The flow duct 32 can be blocked off from the inlet 30 by an inlet lock 42 and from the outlet duct 36 by an outlet lock 44.

The device 10b is shown in the collection mode in FIG. 2a and in the detection mode in FIG. 2b. In the collection mode according to FIG. 2a, the inlet lock 42 is open, in such a way that the inlet 30 communicates with the flow duct 32. The outlet lock 44, which alternately locks either the outlet duct 36 or the circulation duct 34, is located in the position in which it locks the circulation duct 34. By operating the suction pump 38, a gas flow 46a (preferably an ambient air flow) is sucked into the inlet 30, from where it is passed through the microfilter 12, the flow duct 32 and the outlet duct 36 and guided to a gas outlet 48. In this context, the explosive-substance particles which are transported with the gas flow 46a are suspended on the microfilter 12, where they aggregate, as a result of the smaller pore size thereof. Since the outlet lock 44 is locking the circulation duct 34, this is not flowed through.

After a period of a few seconds, when enough explosive-substance particles have aggregated on the microfilter 12, the device switches over to the detection mode shown in FIG. 2b, in which the inlet lock 42 is locked and the outlet lock 44 is relocated into the position in which it locks the outlet duct 36. Further, the suction pump 38 is switched off and the circulation pump 39 is activated instead. In this case, there is a closed annular flow duct, in which the gas flow 46b circulates. At the same time, electric current is passed through the microfilter 12 via contacts 50, in such a way that the microfilter 12 is heated to a temperature at which the explosive-substance particles are desorbed therefrom. While the gas flow 46b is circulating, the explosive-substance particles adhering to the microfilter 12 are desorbed and pass through the detector 40, where they are detected. The circulation pump 39 is operated in such a way that the circulating gas flow 46b passes through the microfilter 12 in the opposite direction from the gas flow 46a in the collection mode.

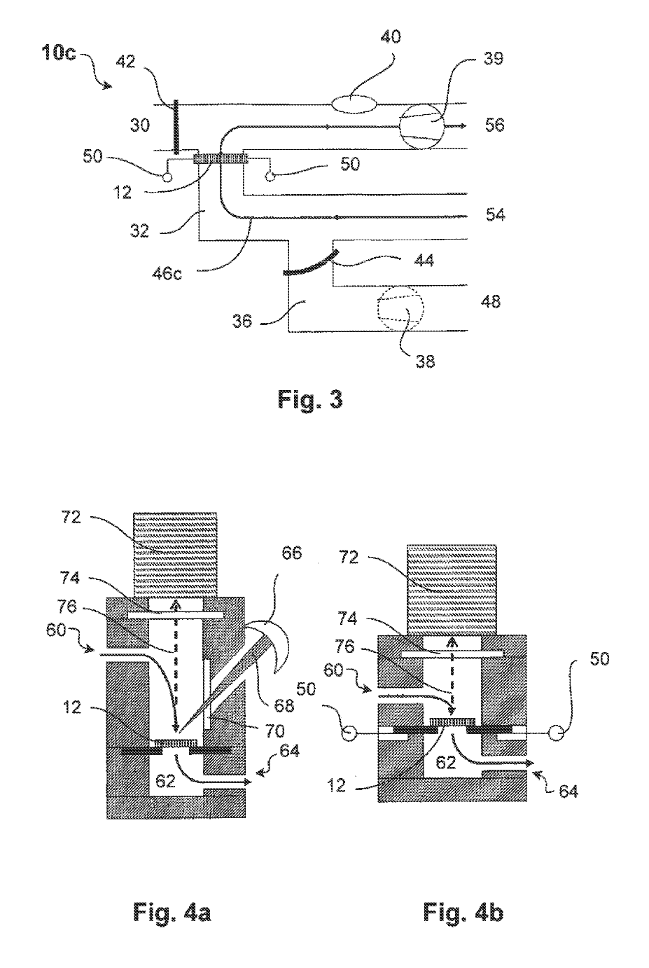

FIG. 3 shows a further embodiment 10c of a detection device, which basically corresponds to the embodiment according to 10b from FIGS. 2a and 2b. By contrast with those embodiments, there is no closed circulation duct, and the flow duct 32 is instead connected to an inlet 54 and an outlet 56. In this embodiment, in the detection mode, instead of being circulated the gas is sucked up via the inlet 54, passed through the microfilter 12, and guided to the outlet 56 by means of the suction pump 39, the explosive-substance particles entrained by the gas flow 46c again being detected by the detector 40. In this context, the microfilter 12 is again heated electrically by means of the terminals 50.

FIGS. 4a and 4b show two embodiments of detection devices comprising heatable microfilters. In the embodiment according to FIG. 4a, a gas inlet 60 opens into a flow duct 62, in which a microfilter 12 is arranged. A gas outlet 64 is provided downstream from the microfilter 12. A halogen lamp 66, preferably of a power of approximately 100 to 200 watts, directs a beam 68 of electromagnetic waves onto the microfilter 12 through a window 70 so as to heat the microfilter 12. An optical thermometer 72 comprising a window 74 is provided so as to detect the heat radiation 76 emitted by the heated microfilter 12 and thus to determine the temperature of the microfilter 12. During operation, the gas which is loaded with explosive-substance particles flows through the gas inlet 60 and the flow duct 62 and passes through the microfilter 12, the explosive-substance particles remaining suspended on the microfilter 12 as a result of the pore size thereof. The gas flow subsequently exits via the gas outlet 64. In this context, the halogen lamp 66 is switched off. This takes place in the collection mode over a period of a few seconds. In the subsequent detection mode, the halogen lamp 66 is activated and heats the microfilter 12, and this is monitored by the thermometer 72. The halogen lamp 66 and temperature sensor 72 are coupled via a control means so as to set a desired temperature or a desired temperature progression of the microfilter 12. In this context, in the detection mode the flow duct 62 can be flowed through in the same flow direction as in the collection mode, as in the embodiment according to FIG. 1, or in the opposite flow direction, as in the embodiments according to FIGS. 2 and 3.

In the embodiment according to FIG. 4b, a resistive heater is provided for the microfilter 12 and is supplied with electrical energy via the terminals 50. This embodiment is simpler in terms of construction, since no optical path is required for the heat radiation. For this purpose, it would be expedient for the microfilter to be fixed to a metal substrate which is heated resistively via electrical contacts. Alternatively, a heating element having surface micromechanics could be structured on the filter, and this would have the advantage of a very low thermal mass and thus of rapid and effective heating and cooling.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B.” Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise.

1-10. (canceled)

11. A method for detecting explosive substance particles in a gas flow, the method comprising:
passing the gas flow through an adsorption net for a specified time period so as to adsorb explosive-substance particles in the gas flow on the adsorption net, the adsorption net including a microfilter having a pore size that is smaller than a particle size of the explosive-substance particles;
heating the adsorption net to a heating temperature so as to desorb the explosive-substance particles from the adsorption net;
supplying a gas flow comprising the desorbed explosive-substance particles to a detector so as to detect the explosive-substance particles.

12. The method according to claim 11, wherein the microfilter has a pore size of less than 1 μm.

13. The method according to claim 12, wherein the microfilter has a pore size of less than 400 nm.

14. The method according to claim 11, wherein the heating temperature is set and the microfilter has a pore size configured such that the explosive-substance particles pass through the microfilter in a gaseous phase after the heating and desorption.

15. The method according to claim 11, wherein the microfilter is heated to a particular temperature so as to detect particular explosive substances.

16. The method according to claim 11, wherein the passing the gas flow through the adsorption net is carried out during a collection mode during which the gas flow moves in a first direction; and wherein the supplying the gas flow including the desorbed explosive-substance particles to the detector is carried out during a detection mode in which the gas flow moves in a second direction that is reverse of the first direction and the gas flow is circulated in a closed circuit so as to flow through the microfilter and pass the detector.

17. A device for detecting explosive-substance particles in a gas flow, the device comprising:
a gas flow path configured to receive a gas flow carrying explosive-substance particles;
a microfilter disposed in the gas flow path and having a particle size that is smaller than a particle size of the explosive-substance particles so as to adsorp the explosive-substance particles thereon when the gas flow passes through the microfilter, the microfilter including a heating device configured to heat the microfilter to a heating temperature so as to desorb the explosive-substance particles from the microfilter;
a detector disposed downstream of the microfilter; and
a control device configured to control a temperature of the microfilter.

18. The device recited in claim 17, wherein the heating device is a halogen lamp that heats the microfilter, and further comprising a temperature sensor configured to detect a temperature of the microfilter.

19. The device recited in claim 17, wherein the heating device heats the microfilter resistively, and further comprising a temperature sensor configured to detect a temperature of the microfilter.

20. A device for detecting explosive-substance particles in a gas flow, the device comprising:
a flow duct configured to receive a gas flow carrying explosive-substance particles;
a microfilter disposed in the flow duct and having a particle size that is smaller than a particle size of the explosive-substance particles so as to adsorp the explosive-substance particles thereon when the gas flow passes through the microfilter;
a circulation duct that is configured to be blocked off from the flow duct during a collection mode and connected to the flow duct during a detection mode so as to form a closed annular duct; and
a detector disposed in the circulation duct.

21. The device recited in claim 20, further comprising a halogen lamp configured to heat the microfilter, and a temperature sensor configured to detect a temperature of the microfilter.

22. The device recited in claim 20, further comprising a heating device that heats the microfilter resistively, and a temperature sensor configured to detect a temperature of the microfilter.

23. A method for producing a microfilter for use in a device for detecting explosive-substance particles in a gas flow, the method comprising forming pores in the microfilter by a photolithography etching process.
